(19) United States
(12) Patent Application Publication
(10) Pub. No.: US 2020/0102272 A1
(43) Pub. Date: Apr. 2, 2020

(54) SYNTHESIS OF SUBSTITUTED ENONES

(71) Applicant: Henkel AG & Co. KGaA, Duesseldorf (DE)

(72) Inventors: Sascha SCHAEFER, Mettmann (DE); Nora SCHMIDT, Hanau (DE); Svenja NEUHAUS, Duisburg (DE)

(21) Appl. No.: 16/576,880

(22) Filed: Sep. 20, 2019

(30) Foreign Application Priority Data
Sep. 28, 2018 (DE) 10 2018 124 026.0

(51) Int. Cl. C07D 207/08

(52) U.S. Cl. CPC C07D 207/08

(57) ABSTRACT

A method for preparing an enone compound of formula (I) is disclosed. Furthermore, the enone compound of formula (I) obtained by the method may be used alone, or in combination with a washing agent, a cleaning agent, an insect repellant agent, a pharmaceutical agent, a fragrance, a pro-fragrance, or combinations thereof.

### CROSS-REFERENCE TO RELATED APPLICATIONS

The present application claims priority to German Patent Application Serial No.: 10 2018 124 026.0 according to 35 U.S.C. § 119, which was filed on Sep. 28, 2018; which is incorporated herein by reference in its entirety and for all purposes.

### TECHNICAL FIELD

The present invention relates to enone synthesis, and more specifically to washing agents, cleaning agents, insect repellants, pharmaceutical compounds, fragrances, pro-fragrances, or combinations thereof comprising substituted enones.

### BACKGROUND

Enones, especially alpha substituted enones, are valuable building blocks for fine and basic chemicals, which are applied in a wide field ranging from pharmaceuticals, fragrances, perfumes, performance polymers, ingredients for washing or cleaning compositions and many others. In particular, enones with a nitrogen bearing substituent in alpha-position are difficult to synthesize. Often, they are only obtainable by complex and multi-step synthesis methods.

Therefore, there is a need for new and simple synthesis methods to produce valuable enones with nitrogen bearing substituents in alpha-position. Especially, a low number of synthesis steps and high atom-economic use of starting materials are desired.

### SUMMARY

Surprisingly, alpha-substituted enones can be produced by direct synthesis of simple amines and ketones in the presence of an oxidant, such as in the presence of a Brønsted acid or Lewis acid, and optionally at elevated temperatures in a non-limiting embodiment.

Therefore, in a first aspect, a method relates to preparing an enone compound of formula (I)

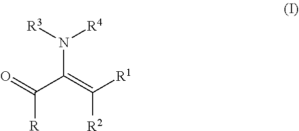

by reacting a ketone compound of formula (II)

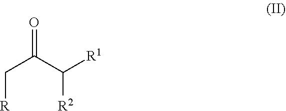

with an amine compound of formula (III)

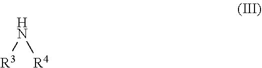

in the presence of at least one oxidant, such as at a temperature in the range of 0 to 300° C., e.g. in the range of 30 to 100° C.;

wherein

(1) R, R¹, R², R³ and R⁴ are independently of each other selected from hydrogen, substituted or unsubstituted linear C1-C20 alkyl groups or branched C3-C20 alkyl groups; substituted or unsubstituted linear C2-C20 alkenyl groups or branched C3-C20 alkenyl groups; substituted or unsubstituted linear C2-C20 alkynyl groups or branched C4-C20 alkynyl groups; substituted or unsubstituted C3-C10 cycloalkyl, C3-C10 cycloalkenyl or aryl groups; substituted or unsubstituted, linear C4-C20 alkylcycloalkyl, C4-C20 alkylcycloalkenyl, C4-C20 alkenylcycloalkyl, C4-C20 alkenylcycloalkenyl, C5-C20 alkynylcycloalkyl or C5-C20 alkynylcycloalkenyl groups, or branched C5-C20 alkylcycloalkyl, C5-C20 alkylcycloalkenyl, C5-C20 alkenylcycloalkyl, C5-C20 alkenylcycloalkenyl, C6-C20 alkynylcycloalkyl or C6-C20 alkynylcycloalkenyl groups, or substituted or unsubstituted linear C7-C20 alkylaryl, C8-C20 alkenylaryl or C8-C20 alkynylaryl groups, or branched C8-C20 alkylaryl, C8-C20 alkenylaryl or C9-C20 alkynylaryl groups; or substituted or unsubstituted linear C1-C20 alkylester, C1-C20 alkylether, or C1-C20 alkylmethoxy, or branched C2-C20 alkylester, C2-C20 alkylether, or C2-C20 alkylmethoxy groups;

wherein each of the groups of R, R¹, R², R³ and R⁴ may further contain 1 to 6 heteroatoms, or

wherein

(2) R and R² and/or R³ and R⁴ each form a substituted or unsubstituted hydrocarbon ring with 3 to 12 carbon atoms, wherein each of the hydrocarbon rings may further contain at least one heteroatom, selected from O, N, S, and P, and/or wherein each of the hydrocarbon rings may further contain at least one double bond; and

wherein the remaining groups are as defined above.

In a second aspect, an enone compound of formula (I) may be obtained by the method described above.

In addition, in a third aspect, a washing or cleaning agent may include the enone compound of formula (I).

Furthermore, in a fourth aspect an insect repellent composition may include the enone compound of formula (I).

In addition, in a fifth aspect, a pharmaceutical composition may include the enone compound of formula (I).

Finally, in a sixth aspect, the enone compound of formula (I) may be used as a fine chemical, a basic chemical, insect repellant, a pharmaceutical compound, a fragrance or a pro-fragrance compound.

### DETAILED DESCRIPTION

“One or more”, as used herein, relates to “at least one” and comprises 1, 2, 3, 4, 5, 6, 7, 8, 9 or more of the referenced species. Similarly, “at least one” means “one or more”, i.e. 1, 2, 3, 4, 5, 6, 7, 8, 9 or more. “At least one”, as used herein in relation to any component, refers to the number of chemically different atoms or molecules, i.e. to the number of different types of the referenced species, but not to the total number of atoms or molecules. For example, “at least one oxidant” means that at least one type of oxidant falling within the definition can be used in the method, but that also two or more different oxidant types falling within this definition can be present, but does not mean that several molecules of only one type of oxidant may be present.

Numeric values specified without decimal places refer to the full value specified with one decimal place. For example, “99%” means “99.0%”, if not stated otherwise.

The expressions “approx.” or “about”, in conjunction with a numerical value, refer to a variance of ±10% relative to the given numerical value, such as ±5%, e.g. ±1%, if not explicitly stated otherwise.

All mol % employed herein relate to the total amount of the respective starting materials, if not explicitly stated otherwise. The amount of the enone compound of formula (I) in washing or cleaning agents is indicated in weight percent (wt.-%), based on the total weight of the respective composition, if not explicitly stated otherwise.

These and other aspects, features and advantages of the non-limiting embodiments become apparent to a person skilled in the art after reading the following detailed description and claims. Each feature from one non-limiting aspect can be used or combined with one or more non-limiting aspects. Furthermore, the examples contained herein are intended to describe and illustrate the non-limiting embodiments, but do not restrict the claims, nor limit the claims to these non-limiting examples.

A method for preparing an enone compound of formula (I)

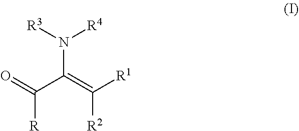

may include reacting a ketone compound of formula (II)

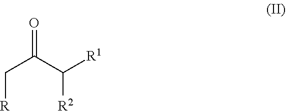

with an amine compound of formula (III)

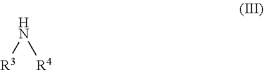

in the presence of at least one oxidant, at a temperature in the range of 0 to 300° C. in a non-limiting embodiment, such as in the range of 30 to 100° C. in an alternative non-limiting embodiment.

(1) R, R¹, R², R³ and R⁴ are, independently of each other, selected from hydrogen; substituted or unsubstituted linear C1-C20 alkyl groups or branched C3-C20 alkyl groups; substituted or unsubstituted linear C2-C20 alkenyl groups or branched C3-C20 alkenyl groups; substituted or unsubstituted linear C2-C20 alkynyl groups or branched C4-C20 alkynyl groups; substituted or unsubstituted C3-C10 cycloalkyl, C3-C10 cycloalkenyl or aryl groups; substituted or unsubstituted, linear C4-C20 alkylcycloalkyl, C4-C20 alkylcycloalkenyl, C4-C20 alkenylcycloalkyl, C4-C20 alkenylcycloalkenyl, C5-C20 alkynylcycloalkyl or C5-C20 alkynylcycloalkenyl groups, or branched C5-C20 alkylcycloalkyl, C5-C20 alkylcycloalkenyl, C5-C20 alkenylcycloalkyl, C5-C20 alkenylcycloalkenyl, C6-C20 alkynylcycloalkyl or C6-C20 alkynylcycloalkenyl groups; or substituted or unsubstituted linear C7-C20 alkylaryl, C8-C20 alkenylaryl or C8-C20 alkynylaryl groups, or branched C8-C20 alkylaryl, C8-C20 alkenylaryl or C9-C20 alkynylaryl groups; or substituted or unsubstituted linear C1-C20 alkylester, C1-C20 alkylether, or C1-C20 alkylmethoxy, or branched C2-C20 alkylester, C2-C20 alkylether, or C2-C20 alkylmethoxy groups;

wherein each of the groups of R, R¹, R², R³ and R⁴ may further contain 1 to 6 heteroatoms, e.g. 1, 2, 3, or 4 heteroatoms selected from O, N, S, and P, or where no heteroatom is present; or

wherein

(2) R and R² and/or R³ and R⁴ each form a substituted or unsubstituted hydrocarbon ring with 3 to 12 carbon atoms, such as 4 to 6 carbon atoms in a non-limiting embodiment, e.g. with 4 or 5 carbon atoms,

wherein each of the hydrocarbon rings may further contain at least one heteroatom, selected from O, N, S, and P, and/or wherein each of the hydrocarbon rings may further contain at least one double bond; and

wherein the remaining groups are as defined above.

In a non-limiting embodiment, R and R² may form a substituted or unsubstituted hydrocarbon ring with 4 to 6 carbon atoms, e.g. with 5 carbon atoms in a non-limiting embodiment, wherein the hydrocarbon ring may contain at least one heteroatom selected from O, N, S, and P, and/or wherein the hydrocarbon ring may further contain at least one double bond.

R¹ may be or include, but is not limited to, a substituted or unsubstituted linear C1-C15 alkyl, C2-C15 alkenyl, C2-C15 alkynyl, C4-C15 alkylcycloalkyl, C4-C15 alkylcycloalkenyl, C1-C15 alkylester, C1-C15 alkylether, or C1-C15 alkylmethoxy group, or branched C3-C15 alkyl, C3-C15 alkenyl, C4-C15 alkynyl, C5-C15 alkylcycloalkyl, C5-C15 alkylcycloalkenyl, C2-C15 alkylester, C2-C15 alkylether, or C2-C15 alkylmethoxy group; alternatively a substituted or unsubstituted linear C1-C8 alkyl, branched C3-C10 alkyl, linear C4-C15 alkylcycloalkenyl, branched C5-C15 alkylcycloalkenyl, linear C1-C15 alkylmethoxy, or branched C2-C15 alkylmethoxy group, wherein R¹ may further contain at least one heteroatom selected from O, N, S, and P; and wherein R³ and R⁴ are as defined in item 1.

In a non-limiting embodiment, R³ and R⁴ may form a substituted or unsubstituted pyrrolidine group.

In a non-limiting embodiment, at least one hydrogen atom of the pyrrolidine ring may be substituted with substituted or unsubstituted linear C1-C15 alkyl, C2-C15 alkenyl, C2-C15 alkynyl, C4-C15 alkylcycloalkyl, C4-C15 alkylcycloalkenyl, C1-C15 alkylester, C1-C15 alkylether, or C1-C15 alkylmethoxy groups, or branched C3-C15 alkyl, C3-C15 alkenyl, C4-C15 alkynyl, C5-C15 alkylcycloalkyl, C5-C15 alkylcycloalkenyl, C2-C15 alkylester, C2-C15 alkylether, or C2-C15 alkylmethoxy groups; alternatively, with substituted or unsubstituted, linear C1-C8 alkyl, branched C3-C10 alkyl, linear C4-C15 alkylcycloalkenyl, branched C5-C15 alkylcycloalkenyl, linear C1-C15 alkylmethoxy, or branched C2-C15 alkylmethoxy groups; or with —CH₂—O—CH₃ in another non-limiting embodiment;

wherein the substituents may further contain at least one heteroatom selected from O, N, S, and P.

In another non-limiting embodiment, the enone compound of formula (I) is an enone compound of formula (IV)

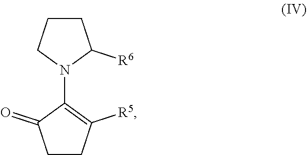

wherein

(1) R⁵ and R⁶ are independently of each other selected from substituted or unsubstituted linear C1-C15 alkyl, C2-C15 alkenyl, C2-C15 alkynyl, C4-C15 alkylcycloalkyl, C4-C15 alkylcycloalkenyl, C1-C15 alkylester, C1-C15 alkylether, or C1-C15 alkylmethoxy groups, or branched C3-C15 alkyl, C3-C15 alkenyl, C4-C15 alkynyl, C5-C15 alkylcycloalkyl, C5-C15 alkylcycloalkenyl, C2-C15 alkylester, C2-C15 alkylether, or C2-C15 alkylmethoxy groups; alternatively, from substituted or unsubstituted, linear C1-C8 alkyl, branched C3-C10 alkyl, linear C4-C15 alkylcycloalkenyl, branched C5-C15 alkylcycloalkenyl, linear C1-C15 alkylmethoxy, or branched C2-C15 alkylmethoxy groups; wherein R⁵ and R⁶ may further contain independently of each other at least one heteroatom selected from O, N, S, and P; or wherein

(2) R⁵ is a substituted or unsubstituted linear C1-C15 alkyl, branched C3-C15 alkyl, linear C4-C15 alkylcycloalkenyl, or branched C5-C15 alkylcycloalkenyl group; and

wherein R⁶ is a substituted or unsubstituted linear C1-C15 alkylmethoxy, or branched C2-C15 alkylmethoxy group, such as —CH₂—O—CH₃ in a non-limiting embodiment.

In another non-limiting embodiment, the enone compound of formula (I) is an enone compound of formula (V) or (VI)

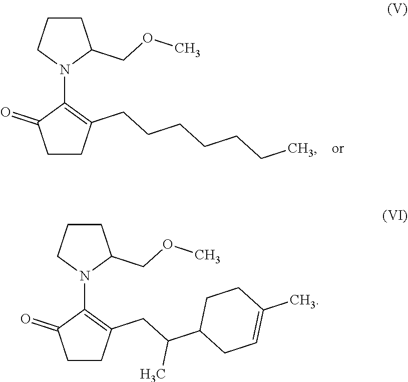

In a non-limiting embodiment, the reaction may be conducted in the presence of at least one Brønsted acid or Lewis acid, e.g. in the presence of para-toluene sulfonic acid.

In a non-limiting embodiment, the reaction may be conducted in the presence of at least one catalyst, such as but not limited to at least one main group metal based catalyst, transition metal based catalyst, organo-catalyst, or mixtures thereof; alternatively, in the presence of at least one transition metal based catalyst, such as but not limited to in the presence of at least one catalyst based on Aluminum (Al), vanadium (V), chrome (Cr), manganese (Mn), iron (Fe), cobalt (Co), nickel (Ni), copper (Cu), zinc (Zn), zirconium (Zr), niobium (Nb), molybdenum (Mo), palladium (Pd), silver (Ag), tungsten (W), iridium (Ir), platinum (Pt), or combinations thereof.

In a non-limiting embodiment, the oxidant is any compound able to act as an oxygen transfer reagent, e.g. oxygen or air, such as air at atmospheric pressure.

In a non-limiting embodiment, at least one catalyst is present in the reaction in an amount ranging from 0.001 to 75 mol %, such as from 0.01 to 5 mol %, based on the total amount of starting materials.

In another non-limiting embodiment, the Brønsted acid(s) or Lewis acid(s) is present in an amount ranging from 0.001 to 75 mol %, such as from 0.01 to 5 mol %, based on the total amount of starting materials.

In a non-limiting embodiment, a washing agent or cleaning agent may include the enone compound of formula (I).

In a non-limiting embodiment, an insect repellant composition may include the enone compound of formula (I).

In a non-limiting embodiment, a pharmaceutical composition may include the enone compound of formula (I).

According to non-limiting embodiments, when one or more groups of R, R¹, R², R³, R⁴, R⁵ and R⁶ are substituted, the substituent can be chosen from every suitable substituent, which is known to the skilled person. In a non-limiting embodiment, each of the groups of R, R¹, R², R³, R⁴, R⁵ and R⁶ may independently of each other contain 1 to 10 of one or more of the following substituents: halogen, ester, ether, hydroxyl, amine, amide, ketone, aldehyde, carboxyl, linear C1-C20 alkyl, branched C3-C20 alkyl, linear C2-C20 alkenyl, branched C3-C20 alkenyl, linear C2-C20 alkynyl, branched C4-C20 alkynyl; C3-C20 cycloalkyl, C3-C20 cycloalkenyl, or aryl; linear C4-C20 alkylcycloalkyl(alkyl), C4-C20 alkylcycloalkenyl(alkyl), C4-C20 alkenylcycloalkyl(alkyl), C4-C20 alkenylcycloalkenyl(alkyl), C5-C20 alkynylcycloalkyl(alkyl) or C5-C20 alkynylcycloalkenyl(alkyl), or branched C5-C20 alkylcycloalkyl(alkyl), C5-C20 alkylcycloalkenyl(alkyl), C5-C20 alkenylcycloalkyl(alkyl), C5-C20 alkenylcycloalkenyl(alkyl), C6-C20 alkynylcycloalkyl(alkyl) or C6-C20 alkynylcycloalkenyl(alkyl); or linear C7-C20 alkylaryl(alkyl), C8-C20 alkenylaryl(alkyl) or C8-C20 alkynylaryl(alkyl), or branched C8-C20 alkylaryl(alkyl), C8-C20 alkenylaryl(alkyl) or C9-C20 alkynylaryl(alkyl), or linear C1-C15 alkylester(alkyl), C1-C15 alkylether(alkyl), or C1-C15 alkylmethoxy groups, or branched C2-C15 alkylester(alkyl), C2-C15 alkylether(alkyl), or C2-C15 alkylmethoxy groups, or combinations thereof. The substituents may contain one or more heteroatom(s), such as, O, N, S, and P. The substituents may replace one or more of —H, —C—, —CH—, —CH₂— and/or —CH₃ in the groups of R, R¹, R², R³, R⁴, R⁵ and R⁶ or in the formed rings of R and R² and/or R³ and R⁴. Non-limiting substituents are linear C1-C10 alkyl, e.g. methyl and ethyl, methylmethoxy (—CH₂—O—CH₃), and linear C4-C20 alkylcycloalkenyl(alkyl) or branched C5-C20 alkylcycloalkenyl(alkyl) groups. Alternatively, the pyrrolidine ring is substituted by a methylmethoxy group (—CH₂—O—CH₃).

The minimum and maximum limit of carbon atoms (CX-CX) of a group of R, R¹, R², R³, R⁴, R⁵ and R⁶ relates to the total amount of carbon atoms of a group of R, R¹, R², R³, R⁴, R⁵ and R⁶ without any optional substituent. That means that optional present substituents of a group of R, R¹, R², R³, R⁴, R⁵ and R⁶ may increase the total number of carbon atoms over the indicated minimum and maximum limit of carbon atoms of the respective group.

The method for preparing an enone compound of formula (I) may be demonstrated in the following non-limiting reaction:

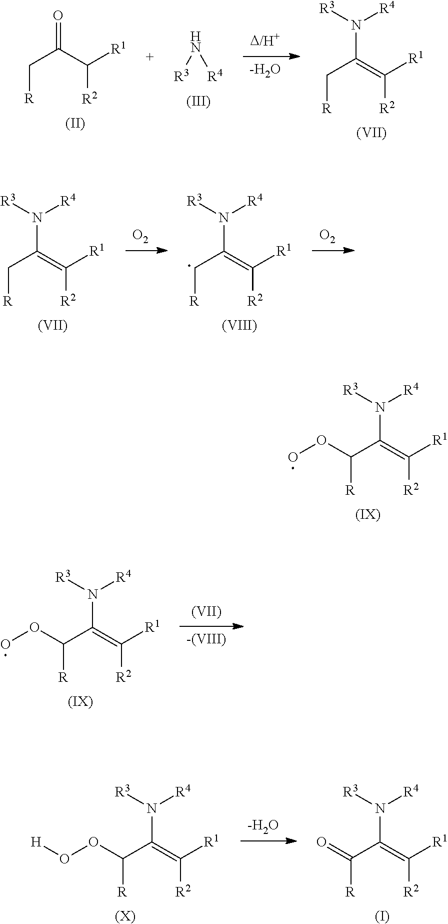

Starting from a ketone compound of formula (II) and an amine compound of formula (III) in the presence of at least one oxidant, the enone compound of formula (I) is formed. Not to be bound to any theory, it is believed that this reaction takes place, since the ketone compound of formula (II) contains a CH₂ group in alpha position to the ketone group.

The reaction is conducted in the presence of an oxidant, which may be any compound able to act as an oxygen transfer reagent, such as oxygen or air, e.g. air at atmospheric pressure.

In another non-limiting embodiment, the reaction is further conducted at temperatures in the range of 0 to 300° C., such as in the range of 30 to 100° C., e.g. in the range of 50 to 80° C.

In a further non-limiting embodiment, the reaction of the method may be conducted in the presence of at least one Brønsted acid or Lewis acid.

Lewis acids are electrophile electron pair acceptors, for example compounds with incomplete or unstable electron octets, such as B(CH₃)₃, BF₃, or AlCl₃, or compounds which are not present in nobel gas configuration. Furthermore, Lewis acids can be metal cations, which can be used as central atom in complex compounds, or molecules with polarized double bonds, such as CO₂ or SO₃, or unsaturated halides, such as SiCl₄.

Brønsted acids are proton donors. These acids release protons to a corresponding base. Non-limiting Brønsted acids are CH₃COOH, H₂SO₄, HSO₄, toluene sulfonic acid, HPO₄²⁻, H₂PO₄⁻, HCl, H₂O, NH₃ and NH₄⁺.

A non-limiting Brønsted acid is para-toluene sulfonic acid.

In a further non-limiting embodiment, the Brønsted acid or Lewis acid is present in amounts of from 0.001 to 75 mol %, such as in amounts of from 0.01 to 5 mol %, based on the total amount of starting materials.

In a further non-limiting embodiment, the reaction is conducted in the presence of at least one catalyst, e.g. in the presence of at least one main group metal based catalyst, transition metal based catalyst, organo catalyst, or mixtures thereof.

Metals, which can be used for main group metal based catalysts are from Groups 1, 2, 13, and 14 (periodic table of elements) and may be in its highest state of oxidation. In non-limiting embodiments, the metal atom is selected from the group comprising lithium (Li), beryllium (Be), sodium (Na), magnesium (Mg), potassium (K), calcium (Ca), boron (B), aluminum (Al), gallium (Ga), indium (In), silicon (Si), germanium (Ge), and tin (Sn). In more non-limiting embodiments, the metal is boron and aluminum.

Suitable transition metal based catalyst, are based on transition metals from the group of vanadium (V), chrome (Cr), manganese (Mn), iron (Fe), cobalt (Co), nickel (Ni), copper (Cu), zinc (Zn), zirconium (Zr), niobium (Nb), molybdenum (Mo), palladium (Pd), silver (Ag), tungsten (W), iridium (Ir), or platinum (Pt).

Suitable organo catalysts are catalysts, which do not contain metal atoms. In non-limiting embodiments, these catalysts are based on phosphor (P), sulfur (S), nitrogen (N), oxygen (O), carbon (C) and hydrogen (H).

In non-limiting embodiments, the at least one catalyst used in the reaction is a transition metal based catalyst.

Suitable catalysts are for example solid supported Cu-catalysts as disclosed in RSC Advances, 5(106), 87221-87227; 2015, silica supported strong Lewis acid AlCl₃ for example disclosed in RSC Advances, 6(14), 11528-11535; 2016); Pd₂(dba)₃ via a triflate for example disclosed in Macromolecular Chemistry and Physics, 211(21), 2339-2346; 2010, Co(OAc)₂ on 1,4 benzochinone for example disclosed in Angewandte Chemie, International Edition, 57(31), 9805-9809; 2018.

Further, the catalyst is present in amounts of from 0.001 to 75 mol %, such as in amounts of from 0.01 to 5 mol %, based on the total amount of starting materials.

According to a non-limiting embodiment, the enone compound may have the formula of formula (I)

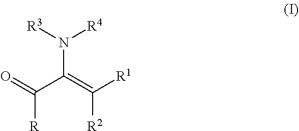

wherein

(1) R, R¹, R², R³ and R⁴ are independently of each other selected from hydrogen, substituted or unsubstituted linear C1-C20 alkyl groups or branched C3-C20 alkyl groups; substituted or unsubstituted linear C2-C20 alkenyl groups or branched C3-C20 alkenyl groups; substituted or unsubstituted linear C2-C20 alkynyl groups or branched C4-C20 alkynyl groups; substituted or unsubstituted C3-C10 cycloalkyl, C3-C10 cycloalkenyl or aryl groups; substituted or unsubstituted, linear C4-C20 alkylcycloalkyl, C4-C20 alkylcycloalkenyl, C4-C20 alkenylcycloalkyl, C4-C20 alkenylcycloalkenyl, C5-C20 alkynylcycloalkyl or C5-C20 alkynylcycloalkenyl groups, or branched C5-C20 alkylcycloalkyl, C5-C20 alkylcycloalkenyl, C5-C20 alkenylcycloalkyl, C5-C20 alkenylcycloalkenyl, C6-C20 alkynylcycloalkyl or C6-C20 alkynylcycloalkenyl groups; or substituted or unsubstituted linear C7-C20 alkylaryl, C8-C20 alkenylaryl or C8-C20 alkynylaryl groups, or branched C8-C20 alkylaryl, C8-C20 alkenylaryl or C9-C20 alkynylaryl groups; or substituted or unsubstituted linear C1-C20 alkylester, C1-C20 alkylether, or C1-C20 alkylmethoxy, or branched C2-C20 alkylester, C2-C20 alkylether, or C2-C20 alkylmethoxy groups;

wherein each of the groups of R, R¹, R², R³ and R⁴ may further contain 1 to 6 heteroatoms, such as 1, 2, 3, or 4 heteroatoms selected from O, N, S, and P; or wherein

(2) R and R² and/or R³ and R⁴ each form a substituted or unsubstituted hydrocarbon ring with 3 to 12 carbon atoms, such as 4 to 6 carbon atoms, alternatively from 4 or 5 carbon atoms,

wherein each of the hydrocarbon rings may further contain at least one heteroatom, selected from O, N, S, and P, and/or wherein each of the hydrocarbon rings may further contain at least one double bond; and

wherein the remaining groups are as defined above.

In a non-limiting embodiment, R and R² of the enone compound of formula (I) form a substituted or unsubstituted hydrocarbon ring with 4 to 6 carbon atoms, e.g. with 5 carbon atoms, wherein the hydrocarbon ring may contain at least one heteroatom selected from O, N, S, and P, and/or wherein the hydrocarbon ring may further contain at least one double bond; and

wherein R¹ is a substituted or unsubstituted linear C1-C15 alkyl, C2-C15 alkenyl, C2-C15 alkynyl, C4-C15 alkylcycloalkyl, C4-C15 alkylcycloalkenyl, C1-C15 alkylester, C1-C15 alkylether, or C1-C15 alkylmethoxy group, or branched C3-C15 alkyl, C3-C15 alkenyl, C4-C15 alkynyl, C5-C15 alkylcycloalkyl, C5-C15 alkylcycloalkenyl, C2-C15 alkylester, C2-C15 alkylether, or C2-C15 alkylmethoxy group; alternatively, a substituted or unsubstituted linear C1-C8 alkyl, branched C3-C10 alkyl, linear C4-C15 alkylcycloalkenyl, branched C5-C15 alkylcycloalkenyl, linear C1-C15 alkylmethoxy, or branched C2-C15 alkylmethoxy group, wherein R¹ may further contain at least one heteroatom selected from O, N, S, and P.

In a non-limiting embodiment, R¹ is substituted or unsubstituted linear C1-C10 alkyl or branched C3-C10 alkyl, substituted or unsubstituted linear C4-C15 alkylcycloalkenyl or branched C5-C15 alkylcycloalkenyl. Alternatively, R¹ is unsubstituted linear C1-C10 alkyl or branched C5-C15 alkylcycloalkenyl(alkyl), e.g. 2-(4-methylcyclohex-3-enyl)propyl.

In another non-limiting embodiment, R³ and R⁴ of the enone compound of formula (I) form a substituted or unsubstituted pyrrolidine group;

wherein at least one hydrogen atom of the pyrrolidine ring is substituted with substituted or unsubstituted linear C1-C15 alkyl, C2-C15 alkenyl, C2-C15 alkynyl, C4-C15 alkylcycloalkyl, C4-C15 alkylcycloalkenyl, C1-C15 alkylester, C1-C15 alkylether, or C1-C15 alkylmethoxy groups, or branched C3-C15 alkyl, C3-C15 alkenyl, C4-C15 alkynyl, C5-C15 alkylcycloalkyl, C5-C15 alkylcycloalkenyl, C2-C15 alkylester, C2-C15 alkylether, or C2-C15 alkylmethoxy groups; alternatively, with substituted or unsubstituted, linear C1-C8 alkyl, branched C3-C10 alkyl, linear C4-C15 alkylcycloalkenyl, branched C5-C15 alkylcycloalkenyl, linear C1-C15 alkylmethoxy, or branched C2-C15 alkylmethoxy groups; e.g. with —CH₂—O—CH₃, wherein the at least one substituent may further contain at least one heteroatom selected from O, N, S, and P.

In another non-limiting embodiment, the enone compound of formula (I) may be an enone compound of formula (IV)

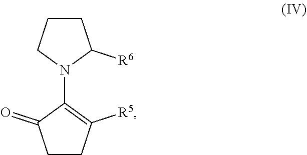

wherein

(1) R⁵ and R⁶ are independently of each other selected from substituted or unsubstituted linear C1-C15 alkyl, C2-C15 alkenyl, C2-C15 alkynyl, C4-C15 alkylcycloalkyl, C4-C15 alkylcycloalkenyl, C1-C15 alkylester, C1-C15 alkylether, or C1-C15 alkylmethoxy groups, or branched C3-C15 alkyl, C3-C15 alkenyl, C4-C15 alkynyl, C5-C15 alkylcycloalkyl, C5-C15 alkylcycloalkenyl, C2-C15 alkylester, C2-C15 alkylether, or C2-C15 alkylmethoxy groups; alternatively, from substituted or unsubstituted, linear C1-C8 alkyl, branched C3-C10 alkyl, linear C4-C15 alkylcycloalkenyl, branched C5-C15 alkylcycloalkenyl, linear C1-C15 alkylmethoxy, or branched C2-C15 alkylmethoxy groups, wherein R⁵ and R⁶ may further contain independently of each other at least one heteroatom selected from O, N, S, and P; or wherein

(2) R⁵ is a substituted or unsubstituted linear C1-C15 alkyl, branched C3-C15 alkyl, linear C4-C15 alkylcycloalkenyl, or branched C5-C15 alkylcycloalkenyl group; and wherein R⁶ is a substituted or unsubstituted linear C1-C15 alkylmethoxy, or branched C2-C15 alkylmethoxy group; e.g. —CH₂—O—CH₃.

In another non-limiting embodiment, the enone compound of formula (I) may be an enone compound of formula (V) or (VI)

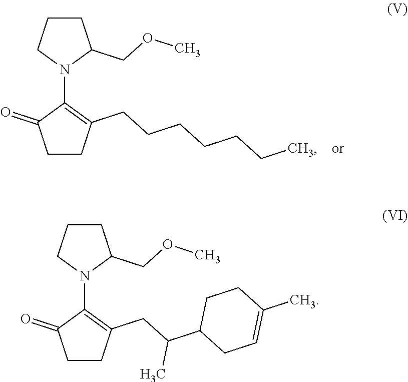

The washing or cleaning agent can be a solid or liquid composition at 20° C. and atmospheric pressure.

In non-limiting embodiments, washing or cleaning agents comprise laundry detergents, laundry after treatment agents, home care products, such as dishwashing detergent, floor cleaning agent or window cleaner, air care products, or cosmetic products.

The washing or cleaning agents may contain at least one further component, such as but not limited to anionic surfactants, cationic surfactants, amphoteric surfactants, nonionic surfactants, acidifiers, alkalizing agents, anti-crease compounds, antibacterial/antimicrobial substances, antioxidants, anti-deposition agents, antistatic agents, bitter substances, bleaching agents, bleach activators, bleach stabilizers, bleach catalysts, builder substances, corrosion inhibitors, ironing aids, co-builders, further fragrances or pro-fragrances, shrinkage inhibitors, electrolytes, emulsifiers, enzymes, enzyme stabilizers, protease inhibitors, colorants, dyes, dye transfer inhibitors, fluorescent agents, fungicides, germicides, metal sequestering agents, odor-complexing substances, auxiliary agents, hydrotropics, rinse aids, complexing agents, preservatives, optical brighteners, perfume carriers, pearl-luster agents, pH control agents, phobing and impregnating agents, polymers, swelling and sliding fasteners, foam inhibitors, layered silicates, dirt-repellent substances, organic solvents, silicone oils, soil-release active substances, UV-protective substances, viscosity regulators, thickeners, discoloration inhibitors, greying inhibitors, vitamins, fabric softeners and water, with the proviso that the at least one further component is different from the enone compound of formula (I).

In non-limiting embodiments, the enone compound of formula (I) is a fragrance or pro-fragrance compound, when used in washing or cleaning agents.

Pro-fragrance compounds are able to release a fragrance compound, which provides a pleasant scent. For example, the fragrance compound is a ketone or diketone compound derived from formula (II).

In a non-limiting embodiment, the fragrance compound is released from the pro-fragrance compound of formula (I) by contact with water, oxygen, sunlight, elevated temperatures or by contact with the skin.

Suitable amounts of the enone compound of formula (I) and of further components for the compositions are based on the application of the washing or cleaning agent and are known to the skilled person.

If the washing agent is a laundry detergent, the enone compound of formula (I) may be present in the washing agent in amounts ranging from 0.001 to 5 wt.-%, such as from 0.01 to 2 wt.-%, based on the total weight of the composition.

In case the washing or cleaning agent is an air care product, the enone compound of formula (I) may be present in the washing agent in amounts of from 0.001 to 99.9 wt.-%, e.g. from 0.01 to 50 wt.-%, based on the total weight of the composition.

In one non-limiting embodiment when an insect repellant includes the enone compound of formula (I), the amount of the enone compound of formula (I) may range from 0.001 to 99.9 wt.-%, such as from 0.01 to 85 wt.-%, alternatively from 0.1 to 70 wt.-%, or from 1 to 50 wt.-% in another non-limiting embodiment, based on the total weight of the insect repellant composition.

In various embodiments the insect repellent composition can comprise further additives, which are common in the field of insect repellant. For example, the additives which are disclosed for washing or cleaning agents can be employed as well.

When a pharmaceutical composition comprises the enone compound of formula (I), the amount of the enone compound of formula (I) may range from 0.001 to 99.9 wt.-%, such as from 0.01 to 85 wt.-%, alternatively from 0.1 to 50 wt.-%, or from 1 to 15 wt.-%, based on the total weight of the pharmaceutical composition.

In various embodiments the pharmaceutical composition can comprise further additives, which are common in the field of pharmaceutical compositions.

In a non-limiting embodiment, enone compounds of formula (I) are used as fine or basic chemicals. In non-limiting embodiments, they are contained in the chemical composition in amounts of at least 90 weight-%, such as at least 95 wt.-%, alternatively at least 99 wt.-%, alternatively at least 99.5 wt.-%, e.g. of at least 99.9 wt.-%, based on the total weight of the fine or basic chemical.

The chemical composition may be a solid or a liquid composition at 20° C. and atmospheric pressure.

It is understood that all embodiments disclosed herein in relation to the method are similarly applicable to the enone compound of formula (I), to the washing or cleaning agents and to the use of the enone compound of formula (I), and vice versa. Specific embodiments and features of the embodiments are to be seen as disclosed for each and every embodiment so that features disclosed in the specification can be combined with each other with the embodiment still being within the scope of the present disclosure.

In the following examples, preferred embodiments are described but it is understood that the claims are not limited thereto.

### EXAMPLES

### General Procedure

The respective ketone (1 eq.), the respective pyrrolidine (1.9 eq-2.5 eq.), and para-toluene sulfonic acid (0.1 mol %) in toluene (150 mL) were refluxed with azeotropic removal of H₂O in a Dean-Stark trap over time. The reaction mixture was then cooled to room temperature, washed with water and dried over Na₂SO₄, filtered and the solvent was evaporated under reduced pressure. The pure products were isolated via column chromatography (hexane-ethyl acetate-1% triethylamine) unless otherwise noted.

### Example 1: 2-[2-(methoxymethyl)pyrrolidin-1-yl]-3-heptylcyclopent-2-en-1-one

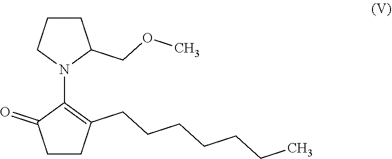 [SMILES: CCCCCCCC1=C(N2CCCC2COC)C(=O)CC1]

2-[2-(methoxymethyl)pyrrolidin-1-yl]-3-heptylcyclopent-2-en-1-one was prepared following the aforementioned method using 2-heptylcyclopentanone (3.27 g, 18 mmol) and 2-methoxymethylpyrrolidine (4.05 g, 35 mmol). Until full conversion of the starting material (approx. 120 hours), the reaction was monitored by gas chromatography (GC).

Pale yellow oil, 11% yield. R_{f}=0.19 (10% ethyl acetate in hexane+1% Et₃N). FTIR (film): \tilde{v}=2924, 2855, 1696, 1635, 1612, 1458, 1376, 1300, 1254, 1221, 1198, 1109, 1014, 972, 916, 819, 724, 675, 573 cm⁻¹. ¹H NMR (400 MHz, CDCl₃): δ=0.89 (t, J=7.0 Hz, 3H), 1.22-1.38 (m, 8H), 1.45-1.55 (m, 2H), 1.64-1.72 (m, 1H), 1.80-1.89 (pseudo-quint, J=6.6 Hz, 7.0 Hz, 2H), 1.99-2.09 (m, 1H), 2.30-2.34 (bd*, J=4.9 Hz, 2H), 2.41-2.48 (m, 4H), 3.02 (q*, J=7.1 Hz, 1H), 3.10 (dd, J=6.9 Hz, 9.3 Hz, 1H), 3.18 (dd, J=4.7 Hz, 9.3 Hz, 1H), 3.26 (q, J=6.3 Hz, 1H), 3.28 (s, 3H), 4.04-4.11 (m, 1H). * shows fine structure.

¹³C-NMR (CDCl₃, 125.8 MHz): δ=14.1 (q), 22.6 (t), 24.5 (t), 27.0 (t), 27.7 (t), 28.9 (t), 29.1 (t), 29.7 (t), 30.7 (t), 31.8 (t), 34.0 (t), 51.9 (t), 57.8 (d), 58.9 (q), 76.3 (t), 144.2 (s), 165.0 (s), 207.5 (s). GC-MS: Optima 5 Accent 815, He, Split 1:10; 50° C.-12° C./min-320° C.; t_{R} =16.1 min. MS (El, 70 eV) m/z (%) at 16.1 min: 293 (1) [M]⁺, 248 (100) [M-C₂H₅O]⁺, 190 (4), 176 (5), 134 (5), 120 (4), 41 (5).

### Example 2: 2-[2-(methoxymethyl)pyrrolidin-1-yl]-3-{2-[2-(4-methylcyclohex-3-en-1-yl)propyl]}cyclo pent-2-en-1-one

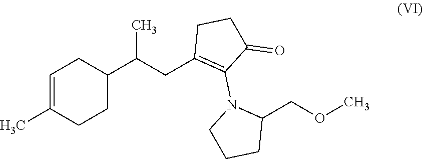

2-[2-(methoxymethyl)pyrrolidin-1-yl]-3-{2-[2-(4-methylcyclohex-3-en-1-yl)propyl]}cyclopent-2-en-1-one was prepared following the aforementioned method using 2-[2-(4-methylcyclohex-3-en-1-yl)propyl]cyclopentanone (6.4 g, 29 mmol) and 2-methoxymethylpyrrolidine (8.0 g, 69 mmol). Until full conversion of the starting material (approx. 175 hours), the reaction was monitored by GC.

Pale yellow oil, 11% yield. R_{f}=0.19 (10% EtOAc in hexane+1% Et₃N). FTIR (film) 2 diastereoisomers: \tilde{v}=2959, 2916, 2872, 2834, 1696, 1633, 1448, 1377, 1198, 1109, 972, 913, 798, 673, 575, 427 cm⁻¹.

¹H NMR (400 MHz, CDCl₃) 2 diastereoisomers: δ=0.82 (d, J=6.8 Hz, 10.2 Hz, isomer A, 3H)/0.84 (d, J=6.8 Hz, 10.2 Hz, isomer B, 3H), 1.18-1.48 (m, 3H), 1.62 (s, 3H), 1.60-1.70 (m, 2H), 1.70-1.88 (m, 2H), 1.84 (t, J=7.1 Hz, 3H), 1.88-2.10 (m, 4H), 2.28-2.52 (m, 6H), 3.02 (q, J=7.1 Hz, 1H), 3.04-3.24 (m, 3H), 3.24 (s, 3H), 4.01-4.09 (m, 1H), 5.36 (bs, 1H).

¹³C-NMR (CDCl₃, 125.8 MHz) 2 diastereoisomers: δ=16.0 (q)/16.2 (q), 23.4 (2 isomers, q), 24.5 (t)/24.6 (t), 24.9 (t), 27.0 (t)/27.2 (t), 27.4/27.5 (t), 29.0 (2 isomers, t), 29.7 (t), 30.7 (t)/30.8 (t), 34.0 (2 isomers, t), 35.0 (t)/35.3 (t), 35.6 (d)/35.8 (d), 38.3 (d)/38.5 (d), 52.0 (2 isomers, t), 57.8 (2 isomers, d), 58.9 (q)/58.9 (q), 76.4 (t)/76.5 (t), 120.7 (2 isomers, d), 133.9 (s)/134.1 (s), 145.4 (s)/145.5 (s), 165.3 (s)/165.7 (s), 207.5 (2 isomers, s). ESI (positive ion) m/z (%): 408 (40), 354 (16), 332 (100) [M+H]⁺, 300 (53). GC-MS: Optima 5 Accent 815, He, Split 1:10; 50° C.-12° C./min-320° C.; t_{R}=18.69 min, 18.76 min. MS (El, 70 eV) m/z (%) for both isomers similar: 331 (6) [M]⁺, 286 (100) [M-C₂H₅O]⁺, 204 (9), 164 (26), 121 (19), 95 (9) [M-C₁₄H₂₂NO₂]⁺, 67 (13), 41 (10).

While specific aspects have been described, it should be understood by those skilled in the art that various changes in form and detail may be made therein without departing from the spirit and scope of the aspects of this disclosure as defined by the appended claims. The scope is thus indicated by the appended claims and all changes that come within the meaning and range of equivalency of the claims are therefore intended to be embraced.

What is claimed is:

1. A method for preparing an enone compound of formula (I) 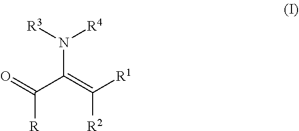
wherein the method comprises:
reacting a ketone compound of formula (II) 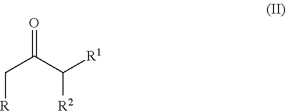
with an amine compound of formula (III) 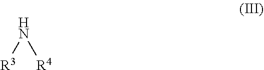
in the presence of at least one oxidant;
wherein
(1) R, R¹, R², R³ and R⁴ are independently of each other selected from hydrogen, substituted or unsubstituted linear C1-C20 alkyl groups or branched C3-C20 alkyl groups; substituted or unsubstituted linear C2-C20 alkenyl groups or branched C3-C20 alkenyl groups; substituted or unsubstituted linear C2-C20 alkynyl groups or branched C4-C20 alkynyl groups; substituted or unsubstituted C3-C10 cycloalkyl, C3-C10 cycloalkenyl or aryl groups; substituted or unsubstituted, linear C4-C20 alkylcycloalkyl, C4-C20 alkylcycloalkenyl, C4-C20 alkenylcycloalkyl, C4-C20 alkenylcycloalkenyl, C5-C20 alkynylcycloalkyl or C5-C20 alkynylcycloalkenyl groups, or branched C5-C20 alkylcycloalkyl, C5-C20 alkylcycloalkenyl, C5-C20 alkenylcycloalkyl, C5-C20 alkenylcycloalkenyl, C6-C20 alkynylcycloalkyl or C6-C20 alkynylcycloalkenyl groups; or substituted or unsubstituted linear C7-C20 alkylaryl, C8-C20 alkenylaryl or C8-C20 alkynylaryl groups, or branched C8-C20 alkylaryl, C8-C20 alkenylaryl or C9-C20 alkynylaryl groups; or substituted or unsubstituted linear C1-C20 alkylester, C1-C20 alkylether, or C1-C20 alkylmethoxy, or branched C2-C20 alkylester, C2-C20 alkylether, or C2-C20 alkylmethoxy groups;
wherein each of the groups of R, R¹, R², R³ and R⁴ optionally contains 1 to 6 heteroatoms; or
wherein
(2) R and R² and/or R³ and R⁴ each form a substituted or unsubstituted hydrocarbon ring with 3 to 12 carbon atoms,
wherein each of the hydrocarbon rings optionally contains at least one heteroatom, selected from O, N, S, and P, and/or wherein each of the hydrocarbon rings optionally contains at least one double bond; and
wherein the remaining groups are as defined above.

2. The method according to claim 1, wherein R and R² form a substituted or unsubstituted hydrocarbon ring with 4 to 6 carbon atoms, wherein the hydrocarbon ring may optionally contains at least one heteroatom selected from O, N, S, and P, and/or wherein the hydrocarbon ring further contains at least one double bond; and
wherein R¹ is a substituted or unsubstituted linear C1-C15 alkyl, C2-C15 alkenyl, C2-C15 alkynyl, C4-C15 alkylcycloalkyl, C4-C15 alkylcycloalkenyl, C1-C15 alkylester, C1-C15 alkylether, or C1-C15 alkylmethoxy group, or branched C3-C15 alkyl, C3-C15 alkenyl, C4-C15 alkynyl, C5-C15 alkylcycloalkyl, C5-C15 alkylcycloalkenyl, C2-C15 alkylester, C2-C15 alkylether, or C2-C15 alkylmethoxy group, wherein R¹ optionally contains at least one heteroatom selected from O, N, S, and P.

3. The method according to claim 1, wherein R³ and R⁴ form a substituted or unsubstituted pyrrolidine group;
wherein at least one hydrogen atom of the pyrrolidine ring is substituted with substituted or unsubstituted linear C1-C15 alkyl, C2-C15 alkenyl, C2-C15 alkynyl, C4-C15 alkylcycloalkyl, C4-C15 alkylcycloalkenyl, C1-C15 alkylester, C1-C15 alkylether, or C1-C15 alkylmethoxy groups, or branched C3-C15 alkyl, C3-C15 alkenyl, C4-C15 alkynyl, C5-C15 alkylcycloalkyl, C5-C15 alkylcycloalkenyl, C2-C15 alkylester, C2-C15 alkylether, or C2-C15 alkylmethoxy groups,
wherein the at least one substituent optionally contains at least one heteroatom selected from O, N, S, and P.

4. The method according to claim 1, wherein the enone compound of formula (I) is an enone compound of formula (IV) 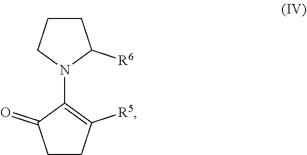
wherein
(1) R⁵ and R⁶ are independently of each other selected from substituted or unsubstituted linear C1-C15 alkyl, C2-C15 alkenyl, C2-C15 alkynyl, C4-C15 alkylcycloalkyl, C4-C15 alkylcycloalkenyl, C1-C15 alkylester, C1-C15 alkylether, or C1-C15 alkylmethoxy groups, or branched C3-C15 alkyl, C3-C15 alkenyl, C4-C15 alkynyl, C5-C15 alkylcycloalkyl, C5-C15 alkylcycloalkenyl, C2-C15 alkylester, C2-C15 alkylether, or C2-C15 alkylmethoxy groups,—wherein R⁵ and R⁶ optionally contains independently of each other at least one heteroatom selected from O, N, S, and P; or
wherein
(2) R⁵ is a substituted or unsubstituted linear C1-C15 alkyl, branched C3-C15 alkyl, linear C4-C15 alkylcycloalkenyl, or branched C5-C15 alkylcycloalkenyl group; and
wherein R⁶ is a substituted or unsubstituted linear C1-C15 alkylmethoxy, or branched C2-C15 alkylmethoxy group.

5. The method according to claim 1, wherein the enone compound of formula (I) is an enone compound of formula (V) or (VI)

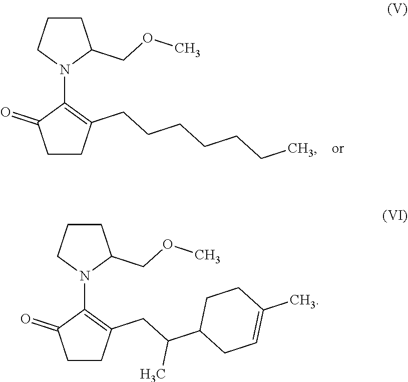

6. The method according to claim 1, wherein the reaction is conducted in the presence of at least one Brønsted acid or Lewis acid.

7. The method according to claim 1, wherein the reaction is conducted in the presence of at least one catalyst.

8. The method according to claim 1, wherein the oxidant is any compound capable of acting as an oxygen transfer reagent.

9. The method according to claim 1, wherein the at least one catalyst is present in an amount ranging from 0.001 to 75 mol based on the total amount of starting materials.

10. The method according to claim 1, wherein the at least one Brønsted acid or Lewis acid is present in an amount ranging from 0.001 to 75 mol % based on the total amount of starting materials.

11. An enone compound of formula (I) obtained by the method according to claim 1.

12. A composition comprising:
an agent selected from the group consisting of a washing agent, a cleaning agent, an insect repellant agent, a pharmaceutical agent, and mixtures thereof; and
an enone compound of formula (I): 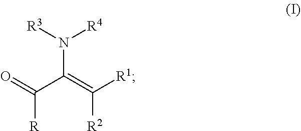
wherein
(1) R, R¹, R², R³ and R⁴ are independently of each other selected from hydrogen, substituted or unsubstituted linear C1-C20 alkyl groups or branched C3-C20 alkyl groups; substituted or unsubstituted linear C2-C20 alkenyl groups or branched C3-C20 alkenyl groups; substituted or unsubstituted linear C2-C20 alkynyl groups or branched C4-C20 alkynyl groups; substituted or unsubstituted C3-C10 cycloalkyl, C3-C10 cycloalkenyl or aryl groups; substituted or unsubstituted, linear C4-C20 alkylcycloalkyl, C4-C20 alkylcycloalkenyl, C4-C20 alkenylcycloalkyl, C4-C20 alkenylcycloalkenyl, C5-C20 alkynylcycloalkyl or C5-C20 alkynylcycloalkenyl groups, or branched C5-C20 alkylcycloalkyl, C5-C20 alkylcycloalkenyl, C5-C20 alkenylcycloalkyl, C5-C20 alkenylcycloalkenyl, C6-C20 alkynylcycloalkyl or C6-C20 alkynylcycloalkenyl groups; or substituted or unsubstituted linear C7-C20 alkylaryl, C8-C20 alkenylaryl or C8-C20 alkynylaryl groups, or branched C8-C20 alkylaryl, C8-C20 alkenylaryl or C9-C20 alkynylaryl groups; or substituted or unsubstituted linear C1-C20 alkylester, C1-C20 alkylether, or C1-C20 alkylmethoxy, or branched C2-C20 alkylester, C2-C20 alkylether, or C2-C20 alkylmethoxy groups;
wherein each of the groups of R, R¹, R², R³ and R⁴ optionally contains 1 to 6 heteroatoms; or
wherein
(2) R and R² and/or R³ and R⁴ each form a substituted or unsubstituted hydrocarbon ring with 3 to 12 carbon atoms,
wherein each of the hydrocarbon rings optionally contains at least one heteroatom, selected from O, N, S, and P, and/or wherein each of the hydrocarbon rings optionally contains at least one double bond; and
wherein the remaining groups are as defined above.
